(19) United States
(12) Patent Application Publication
(10) Pub. No.: US 2025/0143825 A1
(43) Pub. Date: May 8, 2025

(54) SURGICAL INSTRUMENT WITH WRISTED JAWS HAVING IDLER PULLEY FOR ZERO FLEET ANGLE

(71) Applicant: Covidien LP, Mansfield, MA (US)

(72) Inventors: Justin P. Williams, Southbury, CT (US); Joseph T. Eisinger, Northford, CT (US); Michael B. Lyons, Boulder, CO (US); David D. Brause, Longmont, CO (US)

(21) Appl. No.: 18/908,094

(22) Filed: Oct. 7, 2024

Related U.S. Application Data
(60) Provisional application No. 63/572,989, filed on Apr. 2, 2024.
(60) Provisional application No. 63/597,171, filed on Nov. 8, 2023.

(51) Int. Cl. A61B 34/00; A61B 17/29; A61B 34/30

(52) U.S. Cl. CPC A61B 34/71; A61B 17/29; A61B 2017/2903; A61B 2017/2947; A61B 2034/302

(57) ABSTRACT

An end effector for use with a robotic system includes a proximal hub having a first upright support opposing a second upright support, a distal hub having a first upright support and a second upright support and pivotally coupled to the proximal hub about a first pivot axis, a first jaw member, a second jaw member, a first idler pulley, and a second idler pulley. The first and second jaw members are pivotally coupled to the first and second upright supports of the distal hub about a second pivot axis. First and second cable sets are configured to extend around the first and second idler pulleys and the first and second jaw members to rotate the first and second jaw members. The distal hub, the first idler pulley, and the second idler pulley are angled to produce fleet angles of about zero degrees.

### CROSS-REFERENCE TO RELATED APPLICATIONS

The present application claims the benefit of and priority to each of U.S. Provisional Patent Application No. 63/572,989, filed on Apr. 2, 2024, and U.S. Provisional Patent Application No. 63/597,171, filed on Nov. 8, 2023, the entire contents of which are hereby incorporated by reference.

### BACKGROUND

Robotic surgical systems have been used in minimally invasive medical procedures. Some robotic surgical systems include a console supporting a surgical robotic arm and a surgical instrument, having at least one end effector (e.g., forceps, a grasping tool, or a cutting tool), mounted to the robotic arm. The robotic arm provides mechanical power to the surgical instrument for its operation and movement. Each robotic arm may include an instrument drive unit that is operatively connected to the surgical instrument.

Cables extended from the robot console, through the robot arm, and connected to the wrist assembly and/or end effector. In some instances, the cables were actuated by means of motors that were controlled by a processing system including a user interface for a surgeon or clinician to be able to control the robotic surgical system including the robot arm, the wrist assembly and/or the end effector.

In some instances, the wrist assembly provided for articulation of the end effector through the use of cables coupled to different components of the end effector in combination with a pulley system coupled to components of the end effector, such as a surgical tool of the end effector. In these cases, the angle of each cable between the pulleys and the components of the end effector, known as a fleet angle, may be large. Large fleet angles may result in unwanted sliding contact and friction between each cable and pulley or component, thus reducing the longevity of the cable.

Therefore, a need exists for end effectors with components that minimize the fleet angles experienced by articulation cables, thereby increasing the life span of the articulation cables.

### SUMMARY

In accordance with an aspect of this disclosure, an end effector for use with a robotic surgical system is provided. The end effector includes a proximal hub, a distal hub, a first jaw member, a second jaw member, a first idler pulley, and a second idler pulley. The proximal hub includes a first upright support opposing a second upright support. The distal hub includes a first upright support and a second upright support, and is coupled to the first and second upright supports of the proximal hub about a first pivot axis. The first jaw member is pivotally coupled to the first upright support of the distal hub about a second pivot axis. The second jaw member is pivotally coupled to the second upright support of the distal hub about the second pivot axis. The first idler pulley is rotationally coupled to the first upright support of the distal hub. A first cable set is configured to extend around a portion of the first idler pulley and the first jaw member to rotate the first jaw member. The second idler pulley is rotationally coupled to the second upright support of the distal hub. A second cable set is configured to extend around a portion of the second idler pulley and the second jaw member to rotate the second jaw member. The distal hub, the first idler pulley, and the second idler pulley are angled to produce a zero-degree fleet angle.

In an aspect of this disclosure, the end effector may further include a first pulley, a second pulley, a third pulley, and a fourth pulley each coupled to the proximal hub via a distal pulley pin. The first pulley may be disposed adjacent the second upright support of the proximal hub, and the fourth pulley is disposed adjacent the first upright support of the proximal hub. A zero-degree fleet angle may be produced between each of the first idler pulley and the first pulley, the first idler pulley and the second pulley, the second idler pulley and the third pulley, and the second idler pulley and the fourth pulley.

In another aspect of this disclosure, the distal hub may be angled about fifteen degrees relative to the first pulley, the second pulley, the third pulley, and the fourth pulley.

In yet another aspect of this disclosure, the first idler pulley and the second idler pulley may each be angled about ten degrees relative to the first pulley, the second pulley, the third pulley, and the fourth pulley.

In a further aspect of this disclosure, the first jaw member may further include a protrusion. The first cable set may wrap around a portion of the first pulley, a portion of the protrusion of the first jaw member, a portion of the first idler pulley, and a portion of the second pulley.

In an aspect of this disclosure, the second jaw member may further include a protrusion. The second cable set may wrap around a portion of the fourth pulley, a portion of the protrusion of the second jaw member, a portion of the second idler pulley, and a portion of the third pulley.

In another aspect of this disclosure, the end effector may further include a fifth pulley, a sixth pulley, a seventh pulley, and an eighth pulley each coupled to the proximal hub via a proximal pulley pin. The fifth pulley may be disposed adjacent the second upright support of the proximal hub, and the eighth pulley is disposed adjacent the first upright support of the proximal hub.

In yet another aspect of this disclosure, the first jaw member may further include a protrusion. The first cable set may wrap around a portion of the firth pulley, a portion of the first pulley, a portion of the protrusion of the first jaw member, a portion of the first idler pulley, a portion of the second pulley, and a portion of the sixth pulley.

In a further aspect of this disclosure, the second jaw member may further include a protrusion. The second cable set may wrap around a portion of the eighth pulley, a portion of the fourth pulley, a portion of the protrusion of the second jaw member, a portion of the second idler pulley, a portion of the third pulley, and a portion of the seventh pulley.

In an aspect of this disclosure, at least one of the first cable set or the second cable set may include an outer cable portion and an inner cable portion. The inner cable portion of the first cable set or the second cable set may extend around the first idler pulley or the second idler pulley, respectively.

In another aspect of this disclosure, the end effector may define a longitudinal axis. The first pivot axis and the longitudinal axis may define a first plane. The second pivot axis and the longitudinal axis may define a second plane. The second plane may be oriented at a non-orthogonal angle relative to the first plane.

In yet another aspect of this disclosure, the second plane may be angled at about seventy-five degrees relative to the first plane.

In a further aspect of this disclosure, the first idler pulley and the second idler pulley may rotate about a third axis. The third axis and the longitudinal axis may define a third plane oriented at a non-orthogonal angle relative to the first plane.

In an aspect of this disclosure, the third plane may be angled at about eighty degrees relative to the first plane.

In another aspect of this disclosure, the third plane may be angled at about sixty degrees relative to the first plane.

In yet another aspect of this disclosure, an angle of the third plane relative to the first plane may be determined at least in part by a size of at least one of the first idler pulley or the second idler pulley.

In a further aspect of this disclosure, the first upright support of the distal hub may further include a first inner surface and a second inner surface. The first inner surface of the first upright support may be configured to angle the first jaw member and the second inner surface of the first upright support may be configured to angle the first idler pulley. The second upright support of the distal hub may further include a first inner surface and a second inner surface. The first inner surface of the second upright support may be configured to angle the second jaw member and the second inner surface of the second upright support may be configured to angle the second idler pulley.

In an aspect of this disclosure, the first inner surface of the first upright support and the first inner surface of the second upright support may be angled at about fifteen degrees relative to the first plane.

In another aspect of this disclosure, the second inner surface of the first upright support and the second inner surface of the second upright support may be angled at about ten degrees relative to the first plane.

In a further aspect of this disclosure, the second inner surface of the first upright support and the second inner surface of the second upright support may be angled at about thirty degrees relative to the first plane.

According to yet another aspect of this disclosure, an end effector for use with a robotic system, is provided. The end effector includes a proximal hub including a first upright support opposing a second upright support; and a distal hub pivotally coupled to the first and second upright supports of the proximal hub about a first pivot axis. The distal hub includes a first upright support; a second upright support located in juxtaposed relation to the first upright support; and a body portion supporting the first upright support and the second upright support such that a first central plane is defined between the first upright support and the second upright support, the body portion defining a second central plane oriented orthogonal to the first central plane.

The body portion defines a first pair of longitudinally extending passages located on a first side of the distal hub, relative to the first central plane, wherein each passage of the first pair of passages includes a relatively outer inner wall portion facing the second central plane, wherein the outer inner wall portion of each of the first pair of longitudinally extending passages is oriented at an angle relative to the second central plane.

The body portion defines a second pair of longitudinally extending passages located on a second side of the distal hub, opposite the first pair of longitudinally extending passages and opposite the first central plane, wherein each passage of the second pair of passages includes a relatively outer inner wall portion facing the second central plane, wherein the outer inner wall portion of each of the second pair of longitudinally extending passages is oriented at an angle relative to the second central plane.

The end effector further includes a pair of jaw members pivotally coupled to the first upright support and the second upright of the distal hub about a second pivot axis.

### BRIEF DESCRIPTION OF THE DRAWINGS

Embodiments of the present disclosure are described herein with reference to the accompanying drawings, wherein:

FIG. 1 is a schematic illustration of a robotic surgical system including a robotic surgical assembly in accordance with the present disclosure;

FIG. 2A is a perspective view of the robotic surgical assembly and the electromechanical surgical instrument, in accordance with an embodiment of the present disclosure;

FIG. 2B is a perspective view, with parts separated, of the robotic surgical assembly and the electromechanical surgical instrument shown in FIG. 2A;

FIG. 3 is a perspective view of an end effector, having wristed jaws, of the electromechanical surgical instrument for use with the robotic surgical assembly of FIGS. 2A and 2B;

FIG. 4 is a perspective view of the end effector of FIG. 3, showing a cable routed beneath a distal hub of the end effector;

FIG. 5 is a perspective view of the end effector of FIG. 4, with the distal hub hidden to show the cable routing;

FIG. 6 is an enlarged perspective view of the end effector as shown in FIG. 5;

FIG. 7 is a side, perspective view of the end effector of FIG. 6;

FIG. 8 is another side, perspective view of the end effector of FIG. 6, with half of the cable hidden;

FIG. 9 is an alternate side, perspective view of the end effector of FIG. 6;

FIG. 10 is a further alternate side, perspective view of the end effector of FIG. 6;

FIG. 11A is a schematic, distal end view of the end effector illustrated in FIG. 10;

FIG. 11B is another schematic, distal end view of the end effector illustrated in FIG. 10;

FIG. 12 is a distal end view of the distal hub of the end effector illustrated in FIG. 10;

FIG. 13 is a distal end view of an embodiment of the distal hub of the end effector illustrated in FIG. 10;

FIG. 14 is a perspective view of an alternate embodiment of end effector, having wristed jaws, of the electromechanical surgical instrument for use with the robotic surgical assembly of FIGS. 2A and 2B;

FIG. 15 is a cross-section view of the end effector of FIG. 14, as taken through 15-15 of FIG. 14;

FIG. 16 is a perspective view of a yoke of the end effector of FIG. 14;

FIG. 17 is an illustration of cable routing through the yoke of FIG. 16;

FIG. 18 is a perspective view of an alternate yoke of the end effector of FIG. 14; and

FIG. 19 is an illustration of cable routing through the yoke of FIG. 18.

### DETAILED DESCRIPTION

Embodiments of the presently disclosed surgical assembly including an instrument drive unit for driving the operation of an electromechanical surgical instrument and methods thereof are described in detail with reference to the drawings, in which like reference numerals designate identical or corresponding elements in each of the several views. As used herein, the term “distal” refers to that portion of the robotic surgical system, surgical assembly, or component thereof, that is closer to a patient, while the term “proximal” refers to that portion of the robotic surgical system, surgical assembly, or component thereof, that is further from the patient. As used herein, the terms parallel and perpendicular are understood to include relative configurations that are substantially parallel and substantially perpendicular up to about + or −10 degrees from true parallel and true perpendicular.

As used herein, the term “clinician” refers to a doctor, nurse, or other care provider and may include support personnel. In the following description, well-known functions or construction are not described in detail to avoid obscuring the present disclosure in unnecessary detail.

As will be described in detail below, provided is a surgical assembly configured to be attached to a surgical robotic arm. The surgical assembly includes an instrument drive unit having, for example, but not limited to, a motor configured to actuate an end effector of an electromechanical instrument. Operation of the electromechanical instrument may be achieved with, for example, a canister motor (brushless or brushed), via a transmission (gear, belt and/or cable), via pneumatics, and/or via hydraulics. The electromechanical instrument may be driven along an axis of rotation of the electromechanical instrument, which may be integral to the instrument drive unit or to the robotic arm.

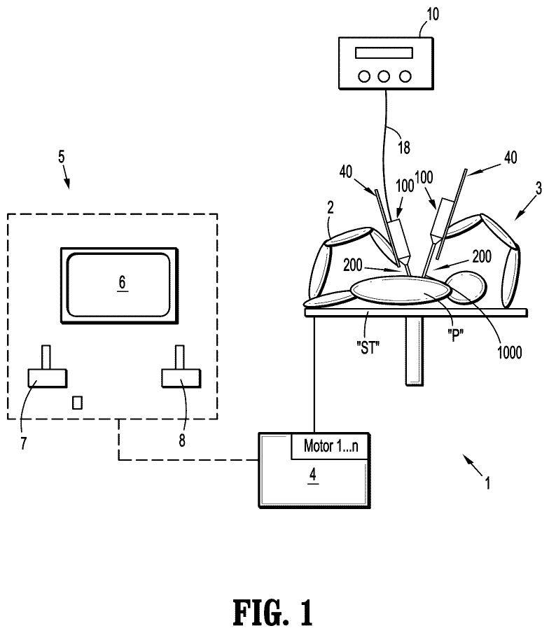

Referring initially to FIG. 1, a surgical system, such as, for example, a robotic surgical system 1, generally includes one or more surgical robotic arms 2, 3, a control device 4, and an operating console 5 coupled with control device 4. Any of the surgical robotic arms 2, 3 may have a robotic surgical assembly 100 and an electromechanical surgical instrument 200 coupled thereto. The electromechanical surgical instrument 200 includes an end effector 1000 disposed at a distal portion thereof. In some embodiments, the robotic surgical assembly 100 may be removably attached to a slide rail 40 of one of the surgical robotic arms 2, 3. In certain embodiments, the robotic surgical assembly 100 may be fixedly attached to the slide rail 40 of one of the surgical robotic arms 2, 3.

Operating console 5 includes a display device 6, which is set up to display three-dimensional images; and manual input devices 7, 8, by means of which a clinician (not shown), is able to telemanipulate the robotic arms 2, 3 in a first operating mode, as known in principle to a person skilled in the art. Each of the robotic arms 2, 3 may be composed of any number of members, which may be connected through joints. The robotic arms 2, 3 may be driven by electric drives (not shown) that are connected to control device 4. The control device 4 (e.g., a computer) is set up to activate the drives, for example, by means of a computer program, in such a way that the robotic arms 2, 3, the attached robotic surgical assembly 100, and thus the electromechanical surgical instrument 200 (including the end effector 1000) execute a desired movement according to a movement defined by means of the manual input devices 7, 8. The control device 4 may also be set up in such a way that it regulates the movement of the robotic arms 2, 3 and/or of the drives.

The robotic surgical system 1 is configured for use on a patient “P” positioned (e.g., lying) on a surgical table “ST” to be treated in a minimally invasive manner by means of a surgical instrument, e.g., the electromechanical surgical instrument 200 and more specifically the end effector 1000 of the electromechanical surgical instrument 200. The robotic surgical system 1 may also include more than two robotic arms 2, 3, the additional robotic arms likewise connected to the control device 4 and telemanipulatable by means of the operating console 5. A surgical instrument, for example, the electromechanical surgical instrument 200 (including the end effector 1000 thereof), may also be attached to any additional robotic arm(s).

The control device 4 may control one or more motors, e.g., motors (not shown), each motor configured to drive movement of the robotic arms 2, 3 in any number of directions. Further, the control device 4 may control an instrument drive unit 110 including motors 52a, 52b, and 52c of a motor pack 50 disposed within a sterile barrier housing 130 of the robotic surgical assembly 100. The motors 52a, 52b, and 52c of the motor pack 50 drive various operations of the end effector 1000 of the electromechanical surgical instrument 200. The motors 52a, 52b, and 52c may include a rotation motor, such as, for example, a canister motor. One or more of the motors 52a, 52b, and 52c (or a different motor, not shown) may be configured to drive a relative rotation of the electromechanical surgical instrument 200, or components thereof, along a longitudinal axis thereof. In some embodiments, each motor 52a, 52b, and 52c of motor pack 50 can be configured to actuate (e.g., rotate) respective drive screws (or, for example, a linear drive, a capstan, etc.) which is operatively connected to a drive rod or a lever arm to effect operation and/or movement of the electromechanical end effector 1000 of the electromechanical surgical instrument 200. In further embodiments, the instrument drive unit 110 may include additional motors within motor pack 50.

With continued reference to FIG. 1, the robotic surgical system 1 includes the robotic surgical assembly 100 that is coupled with or to the robotic arm 2 or 3, and the electromechanical surgical instrument 200 that is coupled to the robotic surgical assembly 100. The robotic surgical assembly 100 transfers power and actuation forces from its motors to driven members of the electromechanical surgical instrument 200 to ultimately drive movement of components of the end effector 1000 of electromechanical surgical instrument 200, for example, an articulation/rotation/pitch/yaw of the end effector 1000. The robotic surgical assembly 100 may also be configured for the activation or firing of an electrosurgical energy-based instrument or the like (e.g., cable drives, pulleys, friction wheels, rack and pinion arrangements, etc.).

As described above, instrument drive unit 110 of robotic surgical assembly 100 includes motor pack 50 and sterile barrier housing 130. Motor pack 50 includes motors 52a, 52b, 52c for controlling various operations of end effector 1000 of electromechanical surgical instrument 200. Electromechanical surgical instrument 200 is removably couplable to instrument drive unit 110 and instrument drive unit 110 is removably couplable or fixedly coupled to slide rail 40 (FIG. 1) of one of the surgical robotic arms 2, 3.

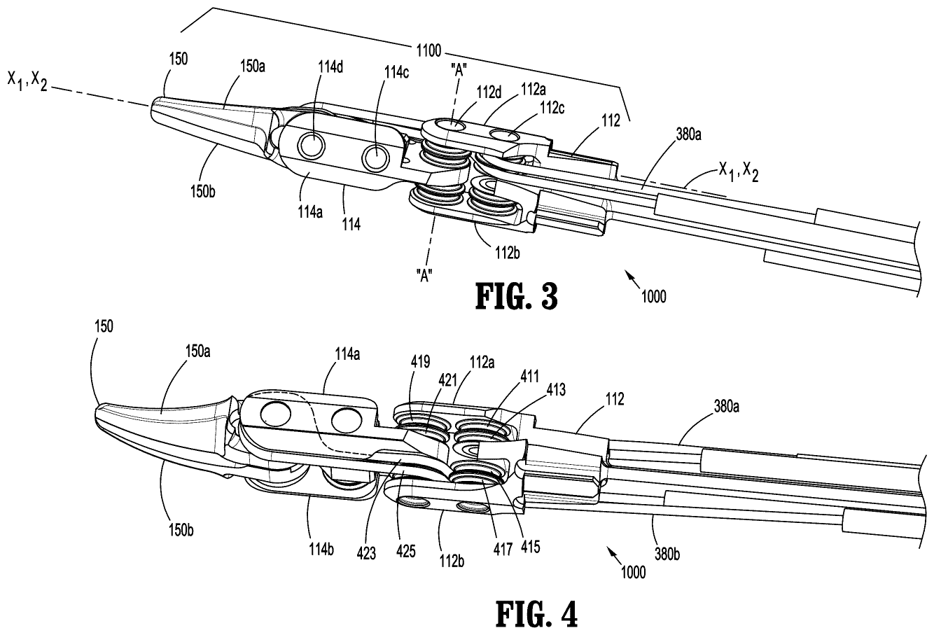

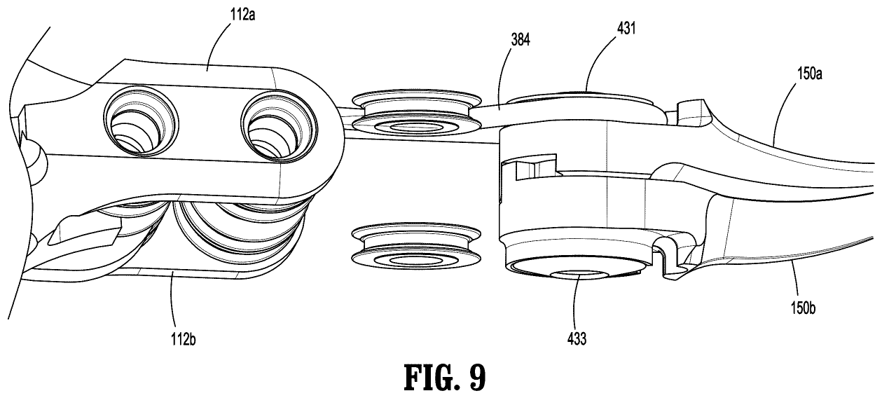

In use, as the motors 52a, 52b, 52c of the motor pack 50 are actuated, rotation of the drive shafts 54a, 54b, 54c of the motors 52a, 52b, 52c, respectively, is transferred to the electromechanical surgical instrument 200. The electromechanical surgical instrument 200 may have a surgical instrument or end effector 1000 (FIGS. 3-9) secured to or securable to a distal end thereof. The electromechanical surgical instrument 200 is configured to transfer rotational forces/movement supplied by the robotic surgical assembly 100 (e.g., via the motors 52a, 52b, 52c of the motor pack 50) into longitudinal movement or translation of cable sets 380a and 380b to effect various functions of the end effector 1000.

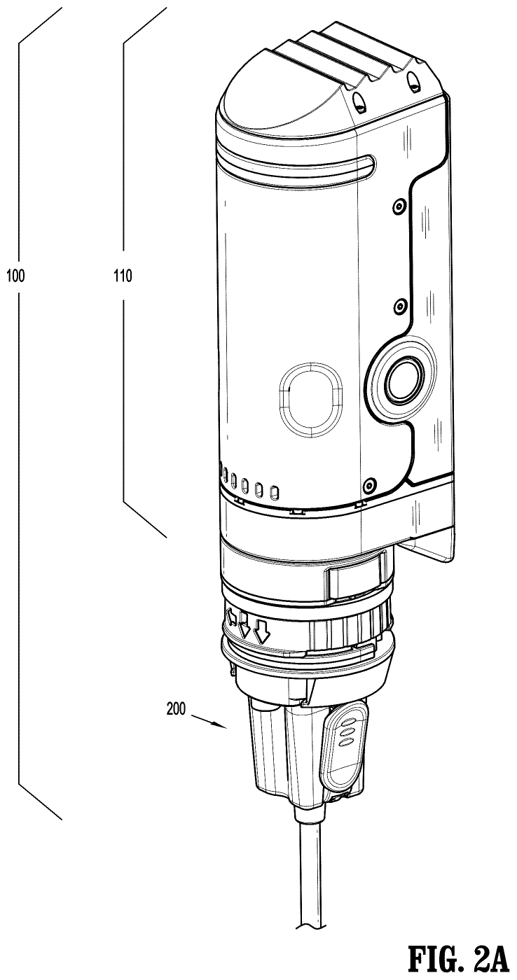

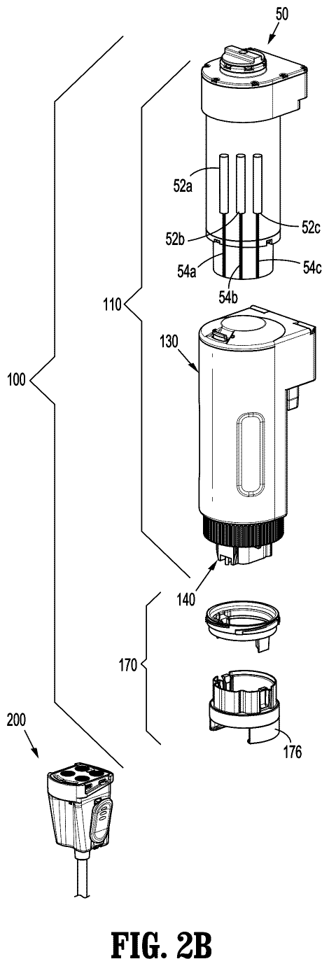

The electromechanical surgical instrument 200 may support an electrical connector configured for selective connection to the plug 140 of the instrument drive unit 110 (FIGS. 2A and 2B) of the robotic surgical assembly 100. The electromechanical surgical instrument 200 may include electronics, including, and not limited to, a memory (for storing identification information, usage information, and the like), wired or wireless communication circuitry (for receiving and transmitting data or information from/to the electromechanical surgical instrument 200, from/to control device 4, and/or from/to a remote central processing system).

Referring now to FIGS. 3-9, an end effector of electromechanical surgical instrument 200 for connection to robot arms 2, 3 and for manipulation by control device 4, will be described and is generally designated as end effector 1000. As described above, end effector 1000 is disposed at a distal portion of electromechanical surgical instrument 200. In one aspect, end effector 1000 may be removably coupled to the distal portion of electromechanical surgical instrument 200 such that a variety of interchangeable end effectors may be used with electromechanical surgical instrument 200. In another aspect, end effector 1000 is fixed and non-removable from the distal portion of the electromechanical surgical instrument.

End effector 1000 is composed of a wrist assembly 1100 and a medical instrument or surgical tool “T.” The wrist assembly 1100 is configured to articulate such that the instrument or surgical tool “T” may be positioned or moved by control device 4 (FIG. 1). Surgical tool “T” may be a jawed instrument (for example, shear tool 150) electrically coupled to an electrosurgical generator 10 (FIG. 1) via a power cable. In certain configurations, a return pad (not shown) may be required which couples a portion of the patient table “ST” or the patient “P” to the electrosurgical generator 10 creating a return path to the electrosurgical generator 10.

Electrosurgical generator 10 is configured to generate electrosurgical radio frequency energy and transmit the generated electrosurgical radio frequency energy to shear tool 150 of end effector 1000 for treatment of tissue via the power cable. It is contemplated that generators such as those sold by Covidien, a division of Medtronic, may be used as a source of electrosurgical energy (electrosurgical generator 10), e.g., Ligasure® Generator, FORCE EZ® Electrosurgical Generator, FORCE FX® Electrosurgical Generator, FORCE 1C™, FORCE 2™ Generator, SurgiStat® II, FORCETRIAD®, VALLEYLAB™ FT10 Energy Platform, and the FORCETRIAD™ Energy Platform electrosurgical generators or other envisioned generators which may perform different or enhanced functions. One such system is described in commonly-owned U.S. Pat. No. 6,033,399, filed on Apr. 9, 1997, entitled “ELECTROSURGICAL GENERATOR WITH ADAPTIVE POWER CONTROL,” the entire content of which is incorporated by reference herein. Further details regarding electrosurgical generator 10 may also be found in U.S. Pat. No. 7,648,499, filed on Mar. 21, 2006, entitled “SYSTEM AND METHOD FOR GENERATING RADIO FREQUENCY ENERGY,” the entire content of which is incorporated by reference herein.

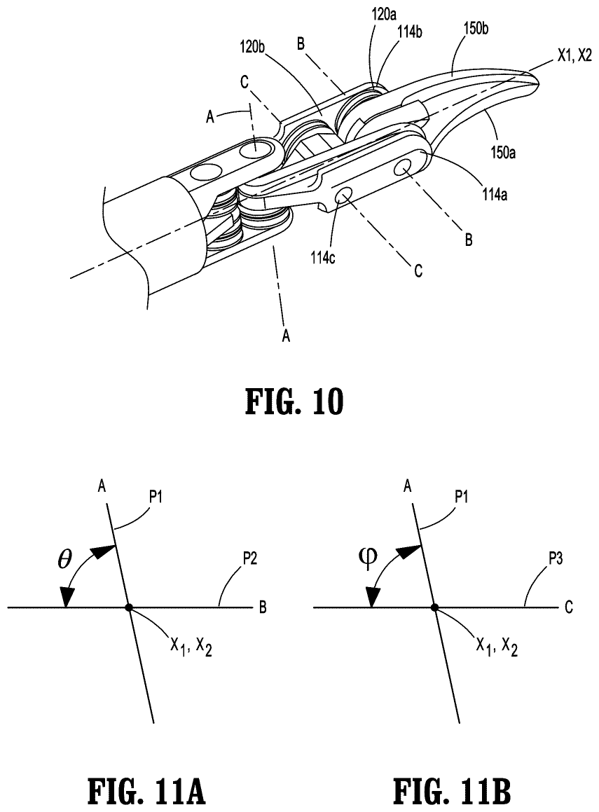

Wrist assembly 1110 of end effector 1000 includes a proximal hub 112, in the form of a distally extending clevis, defining a first longitudinal axis “X1-X1.” Proximal hub 112 defines a first pivot axis “A-A” that is oriented orthogonal to the first longitudinal axis “X1-X1.” In an embodiment, first pivot axis “A-A” may extend through the first longitudinal axis “X1-X1.” First longitudinal axis “X1-X1” and first pivot axis “A-A” define a first plane “P1” (see FIGS. 10 through 13). Proximal hub 112, being in the form of a clevis, includes a pair of spaced apart, opposed upright supports 112a, 112b, a proximal pulley pin 112c, and a distal pulley pin 112d through which first pivot axis “A-A” extends. Wrist assembly 1100 further includes a distal hub 114 (i.e., a yoke) pivotally connected to upright supports 112a, 112b of proximal hub 112 via the distal pulley pin 112d. In particular, a proximal portion of the distal hub 114 is pivotally coupled to the opposed upright supports 112a, 112b of the proximal hub 112 via the distal pulley pin 112d. In this regard, distal hub 114 may pivot relative to proximal hub 112 about first pivot axis “A-A.”

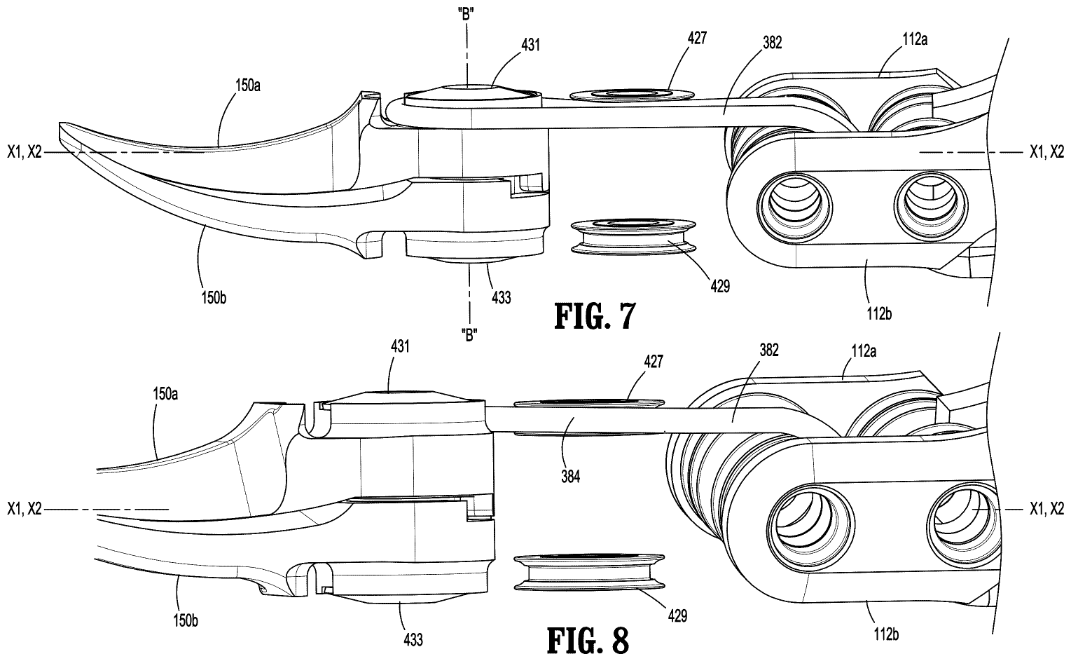

Distal hub 114 may be in the form of a distally extending clevis and defines a second longitudinal axis “X2-X2.” As shown in FIG. 7, distal hub 114 defines a second pivot axis “B-B” that is oriented orthogonal to the second longitudinal axis “X2-X2.” In an embodiment, when the first longitudinal axis “X1-X1” is parallel with the second longitudinal axis “X2-X2” (e.g., electromechanical surgical instrument 200 is in an axially aligned orientation), second pivot axis “B-B” may extend through first longitudinal axis “X1-X1” and the second longitudinal axis “X2-X2.” Further, second longitudinal axis “X2-X2” and second pivot axis “B-B” define a second plane “P2” (see FIGS. 10 and 11A). As illustrated in FIGS. 10 and 11A, second plane “P2” is non-orthogonally angled with respect to first plane “P1”, specifically, second plane “P2” is angled approximately seventy-three to seventy-seven degrees relative to first plane “P1”. For example, second plane “P2” may be angled about seventy-five degrees relative to first plane “P1.” Distal hub 114, being in the form of a clevis, includes a pair of spaced apart, opposed upright supports 114a, 114b, a proximal pulley pin 114c, and a distal hub pin 114d through which second pivot axis “B-B” extends.

Wrist assembly 1100 further includes the jawed instrument, here, shear tool 150, pivotally connected to upright supports 114a, 114b of distal hub 114 via the distal hub pin 114d. In particular a proximal portion of shear tool 150 is pivotally coupled to the opposed upright supports 114a, 114b of the distal hub 114 via the distal hub pin 114d. Shear tool 150 pivots about the second pivot axis “B-B” defined by distal hub 114. Shear tool 150 includes a first jaw member 150a and a second jaw member 150b, each of which is independently pivotable around second pivot axis “B-B” via the distal hub pin 114d.

Continuing with reference to FIGS. 3-9, wrist assembly 1100 of end effector 1000 includes pulley system 400. Pulley system 400 includes pulleys 411, 413, 415, 417, 419, 421, 423, and 425 disposed between upright supports 112a, 112b of proximal hub 112. In particular, in an assembled configuration, pulleys 411, 413, 415, 417 are coupled to upright supports 112a, 112b of proximal hub 112 via proximal pulley pin 112c such that pulleys 411, 413, 415, 417 may spin about proximal pulley pin 112c. In one configuration, pulleys 411, 413 are disposed on one side of a proximal portion of proximal hub 112 and pulleys 415, 417 are disposed on the other side of the proximal portion of proximal hub 112. Additionally, pulleys 419, 421, 423, 425 are coupled to upright supports 112a, 112b of proximal hub 112 via distal pulley pin 112d such that pulleys 419, 421, 423, 425 may spin about distal pulley pin 112d, and pivot the wrist assembly 1100 about axis “A-A.” In one configuration, pulleys 419, 421 are disposed on one side of a distal portion of proximal hub 112 and pulleys 423, 425 are disposed on the other side of the distal portion of proximal hub 112.

Additionally included in pulley system 400 are idler pulleys 427, 429, disposed between upright supports 144a, 114b of distal hub 114. In an assembled configuration, idler pulleys 427, 429 are coupled to upright supports 114a, 114b of distal hub 114 via proximal pulley pin 114c such that pulleys 427, 429 may spin about proximal pulley pin 114c. In one configuration, pulley 427 is disposed on one side of a proximal portion of distal hub 114 and pulley 429 is disposed on the other side of the proximal portion of distal hub 114.

As previously noted, shear tool 150 is pivotally connected to upright supports 114a, 114b of distal hub 114 via the distal hub pin 114d. In particular, first jaw member 150a and second jaw member 150b are coupled to upright supports 114a, 114b of distal hub 114 via distal hub pin 114d such that first and second jaw members 150a, 150b may pivot about distal hub pin 114d, and therefore pivot about axis “B-B” (FIG. 7). In one configuration, first jaw member 150a is disposed on one side of a distal portion of distal hub 114 and second jaw member 150b is disposed on the other side of the distal portion of distal hub 114.

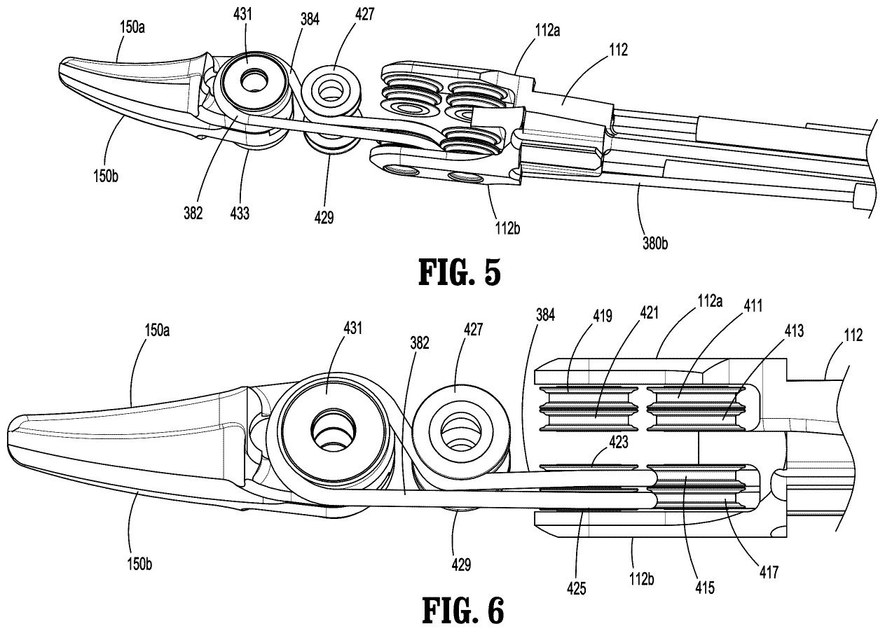

Cable sets 380a, 380b may extend from a portion of the electromechanical surgical instrument 200 to the end effector 1000. Each of cable sets 380a, 380b may be comprised of one or more cables. For example, as will later be described in more detail referencing FIGS. 5-9, cable set 380a may be comprised of cables 382, 384. Each of cable sets 380a, 380b may extend from electromechanical surgical instrument 200 through one or more cable tubes. Cable set 380b is fully shown in FIG. 3 while cable set 380a is fully shown in FIG. 4. Cable set 380a and cable set 380b are partially shown in FIGS. 3 and 4, respectively. As shown in FIGS. 3 and 4, cable set 380b extends around a portion of pulleys 411, 413, 419, and 421, while cable set 380a extends around a portion of pulleys 415, 417, 423, and 425. FIG. 4 in particular shows the routing of cable set 380a beneath distal hub 114, as shown using a dashed line.

FIGS. 5-9 show the end effector 1000 with the distal hub 114 hidden from view, to more clearly show the routing of cable set 380a. As shown, an outer cable portion 382 of cable set 380a extends from the electromechanical surgical instrument 200 toward the end effector 1000. Upon reaching end effector 1000, outer cable portion 382 is first routed around a portion of pulley 417, then over a portion of pulley 425, each of which is disposed closest to upright support 112b. Outer cable portion 382 is then routed around a portion of a protrusion 431 of first jaw member 150a. Protrusion 431 may resemble a pulley, and may include a groove or channel for cable set 380a to travel around. An inner cable portion 384 is then routed around a portion of protrusion 431, then around a portion of idler pulley 427. Upon exiting idler pulley 427, inner cable portion 384 is approximately aligned with pulley 423 and extends around a portion of pulley 423. Inner cable portion then extends around a portion of pulley 415 before returning toward electromechanical surgical instrument 200. In aspects, outer cable portion 384 and inner cable portion 384 are part of the same cable. In further aspects, outer cable portion 384 and inner cable portion 384 are separate cables, and may be connected by an engagement member (not shown). The engagement member may be disposed along an outside portion of protrusion 431.

Cable set 380b may mirror the routing of cable set 380a. In aspects, cable set 380b may include an outer cable portion and an inner cable portion, may be a single cable, or may be made up from multiple cables, similar to cable set 380a. Cable set 380b may extend from the electromechanical surgical instrument 200 toward the end effector 1000. Upon reaching end effector 1000, cable set 380b may first extend around a portion of pulley 411, then over a portion of pulley 419, each of which is disposed adjacent upright support 112a. Cable set 380b is then routed around a portion of a protrusion 433 of second jaw member 150b. Protrusion 433 may resemble a pulley, and may include a groove or channel for cable set 380b to travel around. Cable set 380b is then routed around a portion of idler pulley 429. Upon exiting idler pulley 429, cable set 380b is approximately aligned with pulley 421 and extends around a portion of pulley 421. Cable set 380a then extends around a portion of pulley 413 before returning toward electromechanical surgical instrument 200.

As will be described in greater detail below, the idler pulleys 427, 429 and the distal hub 114 (and therefore the shear tool 150) may be angled to produce a fleet angle of about zero degrees off of pulleys 419, 421, 423, and 425. Fleet angle is defined as the angle of a cable as the cable exits a pulley. The fleet angle is measured between a pulley centerline and a centerline of the cable exiting the pulley. The larger the fleet angle, the more sliding contact there is between the cable and the pulley, creating friction and reducing the life of the cable. Therefore, improvements in cable-life may be realized by reducing or eliminating the fleet angle. To reduce the fleet angle, the distal hub 114 and shear tool 150 may be rotated relative to pulleys 419, 421, 423, and 425, and the first idler pulley and second idler pulley may be rotated relative to pulleys 419, 421, 423, and 425.

To more clearly show the angling of shear instrument 150 and idler pulleys 427, 429 with respect to pulleys 419, 421, 423, and 425, FIGS. 7-9 show a side perspective view of the end effector 1000. FIGS. 7-9 include cable set 380a and hide distal hub 114 and cable set 380b for clarity. FIG. 8 additionally hides outer cable portion 382. The distal hub 114 and shear tool 150 may each be rotated about fifteen degrees relative to pulleys 419, 421, 423, and 425. In other words, with reference to FIGS. 10 and 11A, the second plane “P2” may be located at an angle θ relative to the first plane “P1.” For example, angle θ may be about seventy-five degrees, therefore, second plane “P2” may be angled about seventy-five degrees relative to the first plane “P1.” At the aforementioned angle θ, protrusion 431 of first jaw member 150a forms about a zero-degree fleet angle with pulley 425 for outer cable portion 382 of cable set 380a. Similarly, protrusion 433 of second jaw member 150b forms about a zero-degree fleet angle with pulley 419 for cable set 380b.

Each of idler pulleys 427, 429 may then be rotated about ten degrees relative to pulleys 419, 421, 423, and 425 such that approximately a zero-degree fleet angle is formed. That is, as shown by FIGS. 10 and 11B, distal hub 114 may define a third axis “C-C” that is oriented orthogonal to the second longitudinal axis “X2-X2.” In particular, third axis “C-C” may extend through proximal pulley pin 114c of distal hub 114, which holds idler pulleys 427, 429. In an embodiment, when the first longitudinal axis “X1-X1” is parallel with the second longitudinal axis “X2-X2,” third axis “C-C” may extend through first longitudinal axis “X1-X1” and the second longitudinal axis “X2-X2.” Further, second longitudinal axis “X2-X2” and third axis “C-C” define a third plane “P3” (see FIGS. 10 and 11B). In an embodiment, third plane “P3” and second plane “P2” may be angled equally with respect to first plane “P1.” As illustrated in FIGS. 10 and 11B, like second plane “P2,” third plane “P3” is non-orthogonally angled with respect to first plane “P1.” Specifically, third plane “P3” is angled approximately seventy-eight to eighty-two degrees relative to first plane “P1.” Third plane “P3” may be located at an angle ϕ relative to the first plane “P1,” for example, angle ϕ may be about eighty degrees, therefore, second plane “P2” may be angled about eighty degrees relative to the first plane “P1.”

With reference to FIGS. 7-8, each idler pulley 427, 429 includes a respective central axis, wherein the central axes of the idler pulleys 427, 429 are co-axial with one another such that the idler pulleys are oriented parallel to a plane extending between the idler pulleys 427, 429. However, in additional embodiments, it is envisioned that the central axis of each idler pulley 427, 429 may extend at an angle relative to one another (e.g., such that each idler pulley is angled about 5-45° from the plane extending between the idler pulleys, or in other embodiments about 10-30° from the plane, or in yet other embodiments about 25° from the plane). In yet other embodiments, the central axis of each idler pulley 427, 429 may be parallel to but radially offset from one another, may extend through the longitudinal axes “X1-X1” or “X2-X2”, or may be spaced a radial distance from the longitudinal axes “X1-X1” or “X2-X2”.

At the aforementioned angle ϕ, idler pulley 427 is positioned such that, after extending partially around idler pulley 427, cable set 380a is aligned to extend straight onto pulley 423. At the above-noted angle ϕ, after inner cable portion 384 is routed partially around protrusion 431 of first jaw member 150a and idler pulley 427, about a zero-degree fleet angle is achieved as inner cable portion 384 extends from idler pulley 427 to pulley 423. Similarly, idler pulley 429 is positioned such that, after extending partially around idler pulley 429, cable set 380b is aligned to extend straight onto pulley 421. After cable set 380b travels around a portion of protrusion 433 of second jaw member 150b and idler pulley 429, about a zero-degree fleet angle is achieved as cable set 380b extends from idler pulley 429 to pulley 421.

Angles θ, ϕ of second plane “P2” and third plane “P3,” respectively, may vary depending upon a size and shape of idler pulleys 427, 429. In aspects, angles θ, ¢ may further be impacted depending upon a size and shape of protrusions 431, 433 of first and second jaw members 150a, 150b. As shown in FIGS. 10, 12, and 13, in an embodiment, distal hub 114 may include inner surfaces to aid in appropriately angling idler pulleys 427, 429 and protrusions 431, 433 of first and second jaw members 150a, 150b. Upright support 114a may include a first inner surface 118a, which may contact protrusion 431 of first jaw member 150a, as well as a second inner surface 118b, which may contact idler pulley 427. Upright support 114b may include a first inner surface 120a, which may contact protrusion 433 of second jaw member 150b, as well as a second inner surface 120b, which may contact idler pulley 429. Second inner surfaces 118b, 120b may protrude from, recess from, or be equivalent to first inner surfaces 118a, 120a. First inner surfaces 118a, 120a and second inner surfaces 118b, 120b may prevent undesirable variations in the angling of protrusions 431, 433 and idler pulleys 427, 429 during movement of cable sets 380a, 380b, thus enabling smooth rotation of idler pulleys 427, 429.

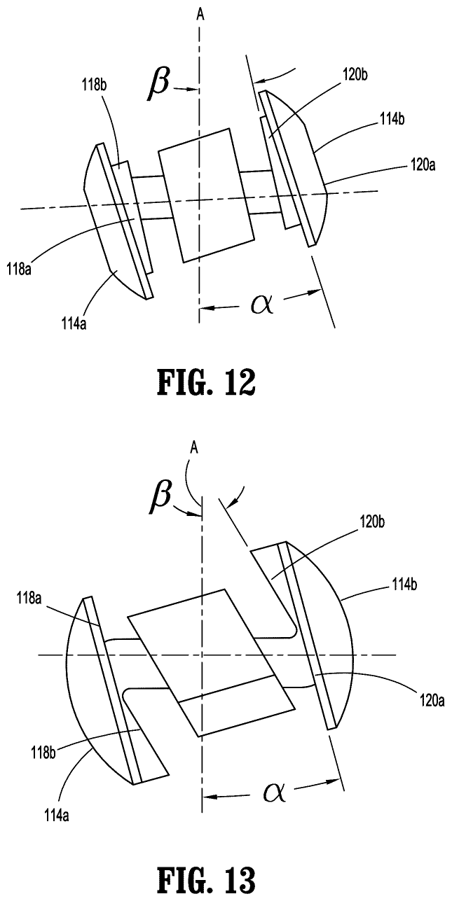

As may be observed particularly in FIGS. 12 and 13, first inner surfaces 118a, 120a may be offset from first plane “P1” at an angle α, and second inner surfaces 118b, 120b may be offset from first plane “P1” at an angle β. Angle α may relate to angle θ, at which second plane “P2” is offset from first plane “P1,” and angle β may relate to angle ϕ, at which third plane “P3” is offset from first plane “P1.” For example, as shown in FIG. 12, angle α of first inner surfaces 118a, 120a may be in a range between thirteen degrees and seventeen degrees offset from first plane “P1,” specifically, angle α may be about fifteen degrees. Accordingly, angle θ of second plane “P2” may be about seventy-five degrees offset from first plane “P1.” Along similar lines, angle β of second inner surfaces 118b, 120b may range between eight and twelve degrees relative to first plane “P1,” specifically, angle β may be about ten degrees. Consequently, angle ϕ of third plane “P3” may be offset about eighty degrees from first plane “P1.” In an embodiment, any of first inner surfaces 118a, 120a or second inner surfaces 118b, 120b may be offset at unique angles from first plane “P1.” Additionally, as opposed to idler pulleys 427, 429 sharing proximal pulley pin 114c and protrusions 431, 433 sharing distal hub pin 114d, any of idler pulleys 427, 429 or protrusions 431, 433 may each be disposed on a respective pin, and thus each may be angled differently relative to first plane “P1.”

It is contemplated that an increase in the size of each of idler pulleys 427, 429 (e.g., an increase in diameter) may result in an increase in angle β. As shown in FIG. 13, angle α may remain at approximately fifteen degrees, with angle θ of second plane “P2” being about seventy-five degrees offset from first plane “P1” as a result. To accommodate larger idler pulleys 427, 429 and maintain approximately a zero-degree fleet angle for cables 380a, 380b between protrusions 431, 433, idler pulleys 427, 429, and pulleys 419, 421, 423, 425, second inner surfaces 118b, 120b may have an angle β ranging between twenty-eight and thirty-two degrees offset from first plane “P1.” In particular, angle β may be approximately thirty degrees, resulting in angle ϕ being about sixty degrees offset from first plane “P1.” It is further contemplated that for relatively decreased idler pulley 427, 429 sizes, angles β, ¢ may be relatively decreased in turn. Other suitable angles θ, ¢, α,β are contemplated for maintaining approximately zero-degree fleet angles through each of cable sets 380a, 380b.

Through movement of cable sets 380a, 380b via electromechanical surgical instrument 200, end effector 1000 may be caused to pivot partially about axis “A-A” (FIG. 3) or may be caused to open and close first and second jaw members 150a, 150b by pivoting each about axis “B-B” (FIG. 7) of wrist assembly 1100. As previously mentioned, cable sets 380a and 380b may comprise two separate cables (for example, outer cable portion 382 and inner cable portion 384 of cable set 380a), or may include a first half and a second half of a single cable. or both sides of the single unitary cable causes corresponding movement of support hub 116. One or both cable sets 380a, 380b or portions thereof may be pulled proximally or advanced distally to affect rotation about axes “A-A” and “B-B.” For example, to pivot end effector 1000 about axis “A-A”, each of cable sets 380a, 380b may be simultaneously advanced or pulled in the same direction. To open or close shear tool 150, thereby pivoting first and second jaw members 150a, 150b apart, each of cable sets 380a, 380b may be advanced or pulled in opposite directions. Movement of cable sets 380a, 380b may be actuated as a result of an input from control device 4 which may activate motors 52a, 52b, 52c.

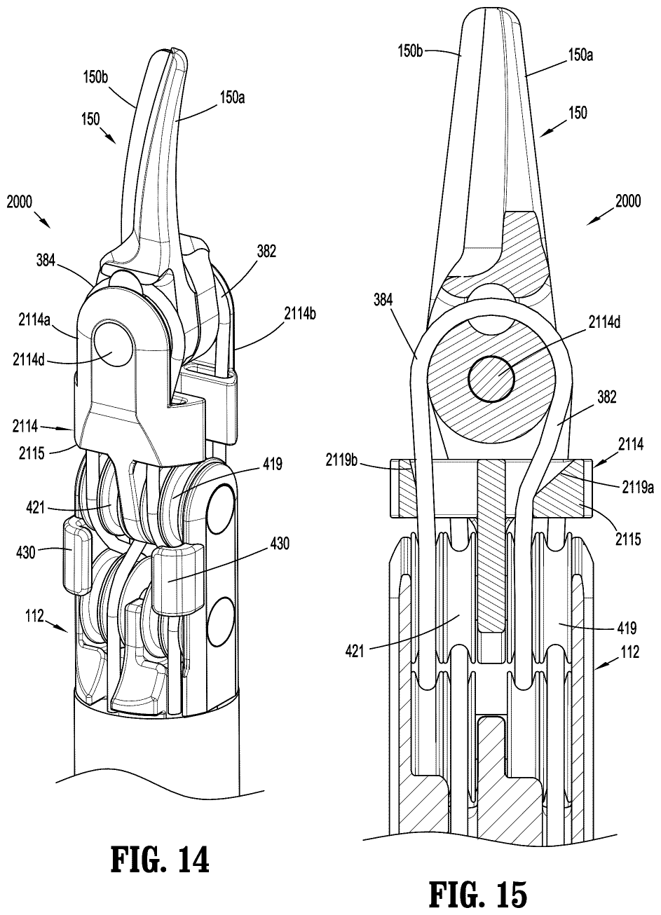

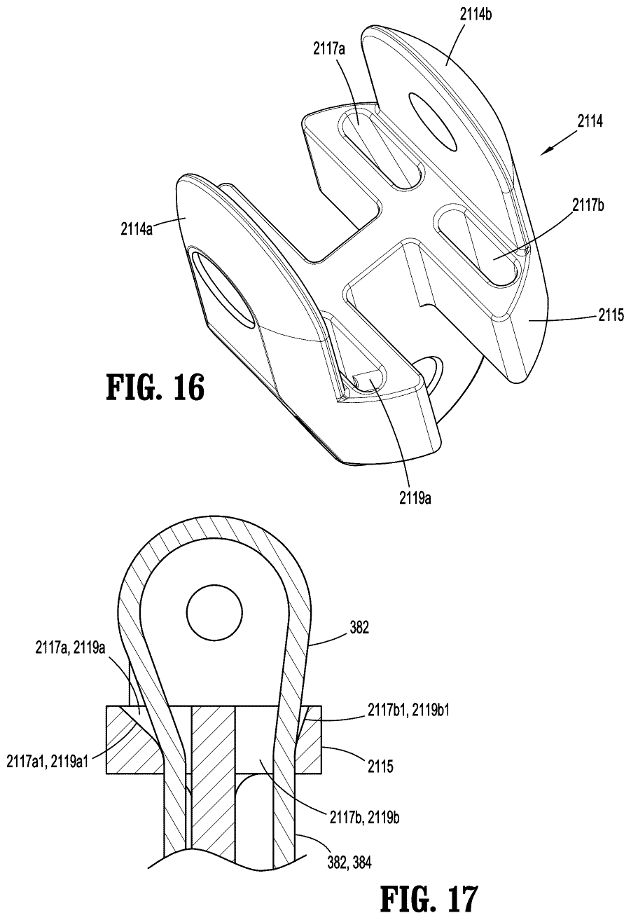

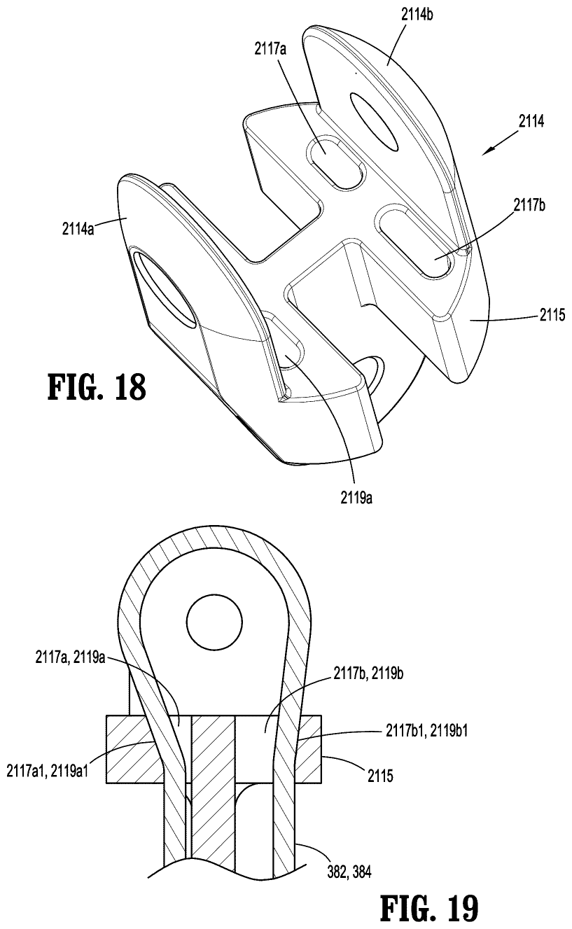

Turning now to FIGS. 14-19, an end effector according to an alternate embodiment of the disclosure is shown as 2000, and will be described. End effector 2000 is substantially similar to end effector 1000, and thus, only differences therebetween will be described hereinbelow. End effector 2000 includes a distal hub or yoke 2114 configured to pivotably support the first and second jaw members 150a, 150b of shear tool 150. Yoke 2114 is pivotally connected to proximal hub 112 of end effector 2000.

Yoke 2114 includes a pair of upright supports 2114a, 2114b extending from a body portion 2115. The pair of upright supports 2114a, 2114b define a first central plane located therebetween and extending along a central longitudinal axis of the end effector 2000, and a second central plane oriented orthogonal to the first central plane. The pair of upright supports 2114a, 2114b of the yoke 2114 support a distal hub pin 2114d on which the first and second jaw members 150a, 150b of shear tool 150 are pivotally supported/connected. The body portion 2115 of the yoke 2114 defines a first pair of longitudinally extending passages 2117a, 2117b located on a first side of the yoke 2114, relative to a central longitudinally extending axis (or the second central plane defined between pair of upright supports 2114a, 2114b) thereof, and a second pair of longitudinally extending passages 2119a, 2119b located on a second side of the yoke 2114, opposite the first pair of longitudinally extending passages 2117a, 2117b.

The first pair of longitudinally extending passages 2117a, 2117b accommodate passage of a cable 382 which cable 382 is secured to, for example, the first jaw member 150a of shear tool 150. The second pair of longitudinally extending passages 2119a, 2119b accommodate passage of another cable 384 which another cable 384 is secured to, for example, the second jaw member 150b of shear tool 150.

Passages 2117a, 2119a of the yoke 2114 each include a respective wall portion 2117a1, 2119a1 located relatively radially outward relative to a plane extending between the pair of passages 2117a, 2117b and between the pair of passages 2119a, 2119b, wherein the wall portions 2117a1, 2119a1 may be disposed at a relatively acute angle relative to the longitudinal axis of the yoke 2114 (as illustrated in FIG. 17), or may be disposed at a relatively shallow angle relative to the longitudinal axis (or the second central plane defined between pair of upright supports 2114a, 2114b) of the yoke 2114 (as illustrated in FIG. 19). The angle of wall portions 2117a1, 2119a1 may be between about 25 degrees to about 50 degrees relative to the longitudinal extending axis (or the second central plane) of the yoke 2114, wherein the relatively shallow angle provides greater surface contact with cables 382 or 384, and wherein the relatively acute angle provides less surface contact with cables 382 or 384.

Passages 2117b, 2119b of the yoke 2114 each include a respective wall portion 2117b1, 2119b1 located relatively radially outward relative to a plane extending between the pair of passages 2117a, 2117b and between the pair of passages 2119a, 2119b, wherein the wall portions 2117b1, 2119b1 may be disposed at a relatively acute angle relative to the longitudinal axis of the yoke 2114 (as illustrated in FIG. 17), or may be disposed at a relatively shallow angle relative to the longitudinal axis of the yoke 2114 (as illustrated in FIG. 19). The angle of wall portions 2117b1, 2119b1 may be between about 5 degrees to about 45 degrees relative to the longitudinal extending axis (or the second central plane) of the yoke 2114, wherein the relatively shallow angle provides greater surface contact with cables 382 or 384, and wherein the relatively acute angle provides less surface contact with cables 382 or 384.

Passages 2117a, 2117b, 2119a, 2119b define wall portions or surfaces 2117a1, 2117b1, 2119a1, 2119b1 against which cables 382, 384 may slide, and provide guiding of cables 382, 384 to/from pulleys 419, 421 of the proximal hub 112 of the end effector 2000. It is envisioned that the passages 2117a, 2117b, 2119a, 2119b of yoke 2114 are axially aligned with respective outer radial surfaces of pulleys 419, 421 so as to minimize or reduce a fleet angle of the cables 382, 384 between yoke 2114 and proximal hub 112.

Additionally, with reference to FIG. 14, each upright support of the proximal hub 112 of the end effector 2000 may include a lip 430 projecting from each side edge thereof (only lips 430 located along one side edge being shown). Each lip 430 projects inwardly towards one another and/or towards the pulleys 419, 421. Also, the lips 430 are located along a length of the side edges of the upright supports of the proximal hub 112 so as to align (axially along the length of the shear tool 150) with the cables 382, 384 extending across and between the yoke 2114 and the proximal hub 112. The lips 430 function to help guide and maintain the cables 382, 384 within the channels of the pulleys 419, 421 and/or prevent or inhibit derailing of the cables 382, 384 from the pulleys 49, 421. Additionally, the lips 430 help to mitigate or reduce fleet angle issues related to the positioning and arrangement of the cables 382, 384 within the wrist assembly of the shear tool 150. In other embodiments, the proximal hub 112 of the end effector 2000 may include only one lip 430 along one side of the upright support or no lips 430 at all.

It will be understood that various modifications may be made to the embodiments disclosed herein. For example, while the cables disclosed herein have been shown and described as being connected to specific portions of the proximal hub, distal hub, and jawed instrument, it is contemplated and within the scope of the present disclosure, for the cables to be operatively connected to any portion of the hubs, supports, or instrument. Therefore, the above description should not be construed as limiting, but merely as exemplifications of various embodiments. Those skilled in the art will envision other modifications within the scope and spirit of the claims appended thereto.

What is claimed is:

1. An end effector for use with a robotic system, the end effector comprising:
a proximal hub including a first upright support opposing a second upright support;
a distal hub pivotally coupled to the first and second upright supports of the proximal hub about a first pivot axis, the distal hub including a first upright support and a second upright support;
a first jaw member pivotally coupled to the first upright support of the distal hub about a second pivot axis;
a second jaw member pivotally coupled to the second upright support of the distal hub about the second pivot axis;
a first idler pulley rotationally coupled to the first upright support of the distal hub, wherein a first cable set is configured to extend around a portion of the first idler pulley and the first jaw member to rotate the first jaw member; and
a second idler pulley rotationally coupled to the second upright support of the distal hub, wherein a second cable set is configured to extend around a portion of the second idler pulley and the second jaw member to rotate the second jaw member,
wherein the distal hub, the first idler pulley, and the second idler pulley are angled to produce a first fleet angle of about zero degrees between the distal hub and the first idler pulley and a second fleet angle of about zero degrees between the distal hub and the second idler pulley.

2. The end effector according to claim 1, further comprising a first pulley, a second pulley, a third pulley, and a fourth pulley each coupled to the proximal hub via a distal pulley pin,
wherein the first pulley is disposed adjacent the second upright support of the proximal hub, and the fourth pulley is disposed adjacent the first upright support of the proximal hub, and
wherein a third fleet angle of about zero degrees is produced between the first idler pulley and the first pulley, a fourth fleet angle of about zero degrees is produced between the first idler pulley and the second pulley, a fifth fleet angle of about zero degrees is produced between the second idler pulley and the third pulley, and a sixth fleet angle of about zero degrees is produced between the second idler pulley and the fourth pulley.

3. The end effector according to claim 2, wherein the distal hub is angled about fifteen degrees relative to the first pulley, the second pulley, the third pulley, and the fourth pulley.

4. The end effector according to claim 2, wherein the first idler pulley and the second idler pulley are each angled about ten degrees relative to the first pulley, the second pulley, the third pulley, and the fourth pulley.

5. The end effector according to claim 2, wherein the first jaw member further comprises a protrusion, and wherein the first cable set wraps around a portion of the first pulley, a portion of the protrusion of the first jaw member, a portion of the first idler pulley, and a portion of the second pulley.

6. The end effector according to claim 2, wherein the second jaw member further comprises a protrusion, and wherein the second cable set wraps around a portion of the fourth pulley, a portion of the protrusion of the second jaw member, a portion of the second idler pulley, and a portion of the third pulley.

7. The end effector according to claim 2, further comprising a fifth pulley, a sixth pulley, a seventh pulley, and an eighth pulley each coupled to the proximal hub via a proximal pulley pin,
wherein the fifth pulley is disposed adjacent the second upright support of the proximal hub, and the eighth pulley is disposed adjacent the first upright support of the proximal hub.

8. The end effector according to claim 7, wherein the first jaw member further comprises a protrusion, and wherein the first cable set wraps around a portion of the firth pulley, a portion of the first pulley, a portion of the protrusion of the first jaw member, a portion of the first idler pulley, a portion of the second pulley, and a portion of the sixth pulley.

9. The end effector according to claim 7, wherein the second jaw member further comprises a protrusion, and wherein the second cable set wraps around a portion of the eighth pulley, a portion of the fourth pulley, a portion of the protrusion of the second jaw member, a portion of the second idler pulley, a portion of the third pulley, and a portion of the seventh pulley.

10. The end effector according to claim 1, wherein at least one of the first cable set or the second cable set includes an outer cable portion and an inner cable portion, and wherein the inner cable portion of the first cable set or the second cable set extends around the first idler pulley or the second idler pulley, respectively.

11. The end effector according to claim 1, wherein:
the end effector defines a longitudinal axis;
the first pivot axis and the longitudinal axis define a first plane;
the second pivot axis and the longitudinal axis define a second plane; and
the second plane is oriented at a non-orthogonal angle relative to the first plane.

12. The end effector according to claim 11, wherein the second plane is angled at about seventy-five degrees relative to the first plane.

13. The end effector according to claim 11, wherein the first idler pulley and the second idler pulley rotate about a third axis, and wherein the third axis and the longitudinal axis define a third plane oriented at a non-orthogonal angle relative to the first plane.

14. The end effector according to claim 13, wherein the third plane is angled at about eighty degrees relative to the first plane.

15. The end effector according to claim 13, wherein the third plane is angled at about sixty degrees relative to the first plane.

16. The end effector according to claim 13, wherein an angle of the third plane relative to the first plane is determined at least in part by a size of at least one of the first idler pulley or the second idler pulley.

17. The end effector according to claim 11, wherein:
the first upright support of the distal hub further includes a first inner surface and a second inner surface, wherein the first inner surface of the first upright support is configured to angle the first jaw member and the second inner surface of the first upright support is configured to angle the first idler pulley, and
the second upright support of the distal hub further includes a first inner surface and a second inner surface, wherein the first inner surface of the second upright support is configured to angle the second jaw member and the second inner surface of the second upright support is configured to angle the second idler pulley.

18. The end effector according to claim 17, wherein the first inner surface of the first upright support and the first inner surface of the second upright support are angled at about fifteen degrees relative to the first plane.

19. The end effector according to claim 17, wherein the second inner surface of the first upright support and the second inner surface of the second upright support are angled at about ten degrees to thirty degrees relative to the first plane.

20. An end effector for use with a robotic system, the end effector comprising:
a proximal hub including a first upright support opposing a second upright support;
a distal hub pivotally coupled to the first and second upright supports of the proximal hub about a first pivot axis, the distal hub including:
a first upright support; and
a second upright support located in juxtaposed relation to the first upright support;
a body portion supporting the first upright support and the second upright support such that a first central plane is defined between the first upright support and the second upright support, the body portion defining a second central plane oriented orthogonal to the first central plane, the body portion defining:
a first pair of longitudinally extending passages located on a first side of the distal hub, relative to the first central plane, wherein each passage of the first pair of passages includes a relatively outer inner wall portion facing the second central plane, wherein the outer inner wall portion of each of the first pair of longitudinally extending passages is oriented at an angle relative to the second central plane; and
a second pair of longitudinally extending passages located on a second side of the distal hub, opposite the first pair of longitudinally extending passages and opposite the first central plane, wherein each passage of the second pair of passages includes a relatively outer inner wall portion facing the second central plane, wherein the outer inner wall portion of each of the second pair of longitudinally extending passages is oriented at an angle relative to the second central plane; and
a pair of jaw members pivotally coupled to the first upright support and the second upright of the distal hub about a second pivot axis.
